(19) United States
(12) Patent Application Publication
(10) Pub. No.: US 2018/0207310 A1
(43) Pub. Date: Jul. 26, 2018

(54) Moisture and Odour Removing Insert

(71) Applicant: 1945975 Alberta Ltd., Sturgeon County (CA)

(72) Inventors: Demetrius John Bazos, Sturgeon County (CA); Peter John Konidas, Edmonton (CA)

(21) Appl. No.: 15/845,715

(22) Filed: Dec. 18, 2017

(30) Foreign Application Priority Data
Jan. 20, 2017 (CA) 2955458

(51) Int. Cl. A61L 9/014; B01J 20/28; B01J 20/02; B01J 20/26; B01J 20/10; B01J 20/34; A61L 9/04; B01D 53/26; B01D 53/04

(52) U.S. Cl. CPC A61L 9/014; B01D 2259/40088; B01J 20/28016; B01J 20/0233; B01J 20/261; B01J 20/264; B01J 20/103; B01J 20/3483; B01J 20/3433; B01J 20/3425; A61L 9/042; B01D 53/261; B01D 53/0407; A61L 2209/22; B01D 2253/106; B01D 2257/80; B01J 20/2805

(57) ABSTRACT

A pod for removing odor and moisture from a substantially confined area has deodorant and moisture adsorbing properties to counteract unpleasant odours, especially odours due to microbial growth. The pod is removably insertable into footwear, gloves, skates, lockers, sports gear, workout gear or any gear or enclosure. The pod has a fabric pouch with anti-microbial properties and contains fragrance-releasing particles and desiccant granules. The moisture adsorbing properties of the pod may be thermally rechargeable. The pods may be attached to other pods in use.

### TECHNICAL FIELD

An insert for removing odor and moisture from a substantially confined area such as a shoe, glove, bag or locker.

### BACKGROUND

It is well known that items like sports gear and work gear can develop an unpleasant smell after use. The items collect moisture due to perspiration during use which can lead to a buildup of bacteria that create unpleasant odours, especially when the items are stored in enclosed containers or bags or left in a humid environment. Aside from unpleasant smells, prolonged exposure to moisture and microbes may also weaken or degrade the materials.

There are a variety of known means for counteracting moisture accumulation and odours caused by bacteria and other microbes in footwear, skates, gloves, lockers, duffel bags, backpacks and other sports or workout gear. However, there remains a need for a removable device to eliminate odours rather than simply masking them. The present device provides active moisture removal, anti-microbial action, a fresh deodorizing scent and the ability to regenerate its moisture removal properties.

### SUMMARY

In an embodiment, there is provided a pod for removing odor and moisture from a substantially confined area comprising a pouch formed from fabric, the fabric incorporating an antimicrobial agent; and desiccant granules and fragrance-releasing particles confined within the pouch.

These and other aspects of the device and method are set out in the claims.

### BRIEF DESCRIPTION OF THE FIGURES

Embodiments will now be described with reference to the figures, in which like reference characters denote like elements, by way of example, and in which:

FIG. 1 is a perspective view of an embodiment of an adsorbent pod.

FIG. 2 is a cross sectional view of the adsorbent pod shown in FIG. 1.

FIG. 3 is a cross sectional view of an embodiment of a multi-compartmented adsorbent pod.

FIG. 4 is an embodiment of an anti-microbial thread where an individual silver strand is woven into the thread.

FIG. 5 is an embodiment of an anti-microbial fabric where silver beads are woven into the fabric.

FIG. 6 is a cross section of an embodiment of an anti-microbial thread coated in silver.

FIG. 7 is a cross section of an embodiment of an anti-microbial fabric coated in silver.

### DETAILED DESCRIPTION

Immaterial modifications may be made to the embodiments described here without departing from what is covered by the claims.

A pod 10 for removing odour and moisture from a substantially confined area has deodorant, moisture adsorbing or absorbing, and anti-microbial properties. The pod 10 may be removably insertable into a substantially confined area or enclosed space, such as footwear, gloves, skates, lockers, sports gear, workout gear or any gear or enclosure susceptible to unpleasant odours. The pods 10 work to remove unpleasant odours, especially those due to microbes including bacteria, by reducing moisture levels and microbial growth within the substantially confined area. The pods may also release a fragrance to mask unpleasant odours. The pods may be inserted in the substantially confined area between uses of the item, and may be used during short- or long-term storage.

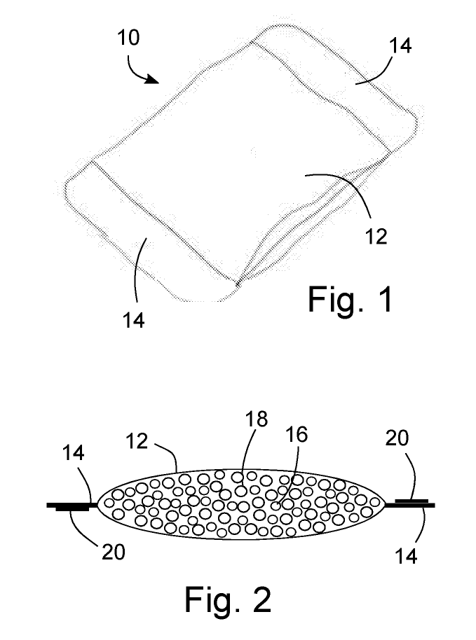

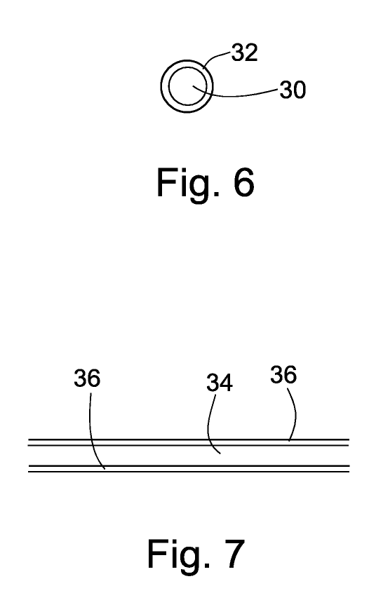

Referring to FIG. 1, the pod 10 has a pouch 12 made of fabric which contains materials with desiccating and fragrant properties. The pouch 12 may be made of any type of cloth or fabric such as a woven synthetic or natural fabric that is permeable to air and water vapour. Preferably, polyester is used to form the pouch. The fabric also preferably includes or is treated with an anti-microbial agent to reduce odours by eliminating their source. The anti-microbial agent may be bonded to the fabric surface after production or bonded directly to or incorporated within the thread prior to production. In an embodiment, the anti-microbial agent is silver. The silver may be incorporated into the fabric in many ways. By way of example only, an individual silver strand 24 may be woven with other strands 22 into an anti-microbial thread which is then used to make the fabric (FIG. 4). The silver may be incorporated as silver beads 26 woven with threads 28 into the fabric (FIG. 5). Silver in the form of a coating 32 may be bonded to polyester thread 30 to form a silver-coated thread (FIG. 6). If silver-coated threads are used, silver-coated and uncoated threads may be woven together to form the anti-microbial fabric, or only silver-coated threads may be used to form the fabric. A silver coating 36 may alternatively be bonded directly to the fabric 34 using a polymer solution (FIG. 7). The fabric should be sufficiently permeable to allow air, moisture and fragrances to pass through the fabric while containing the particles within the pouch. The fabric may form a flexible or rigid pouch.

The pods 10 have a modular design and may be connected to other pods though various attachment mechanisms. For example, the pod may have flaps 14 with complementary Velcro® strips 20 to allow the pod to be joined to other pods. New pods may be added to increase the anti-microbial and moisture absorbing capacities for larger areas. Pods may also be joined to renew the deodorizing scent.

As shown in FIG. 2 (not to scale), the pods are filled with a mixture of desiccant granules 16 and fragrance-releasing particles 18.

The fragrance-releasing particles 18 are used to mask and eliminate unpleasant odors emanating from or within the confined area. The fragrance-releasing particles 18 are composed of a solid carrier which carries an aromatic substance or scent. Many types of aromatic substances may be used, the only limitation being compatibility with the solid carrier. The solid carrier may be formed of a polymer matrix, for example polyethylene glycol (PEG) or ethylene-vinyl acetate (EVA). One or more types of solid carrier may be used in each pouch. The particles may be 2-5 mm in diameter. The PEG may have a molecular weight of 40,000-70,000, and the EVA may have a variable molecular weight dependent on the base material formulation. The aromatic substance may be derived from oils, for example essential oils. The fabric may also be infused with fragrance.

The moisture adsorbing or absorbing properties of the pod are provided by the desiccant granules 16. The desiccant granules 16 may be made of any material with moisture adsorbing or absorbing properties. Preferably, the desiccant granules are made of a material that is capable of adsorbing large amounts of moisture from surrounding air relative to its mass, for example silicon dioxide (or silica gel). Alternative desiccants may be used, preferably ones that are non-toxic, non-corrosive and chemically inert. The granules 16 may be in the form of spheroidal beads or other granules, preferably having a high surface area available for adsorption.

TABLE 1
Example ingredient amounts.

| Weight percentage | Ingredient |
|---|---|
| 1-10% | PEG (polyethylene glycol) |
| 15-40% | EVA (ethylene-vinyl acetate) |
| 10-25% | Scent (eg. an essential oil product) |
| 30-50% | Silicon dioxide |
| 5-15% | Metallic spheres |
| 0-3% | Color |

Table 1 above shows example ranges of the weight percentages of the contents of an embodiment of the pod where the solid carriers are EVA and PEG, the scent or aromatic substance is an essential oil and the desiccant is silicon dioxide. In an embodiment, the amounts of the contents may be (all percentages are weight percentages): 8% PEG, 25% EVA, 11% scent (an essential oil product), 45% silicon dioxide, 10% stainless steel spheres, 1% color. The amount of scent relative to the amount of desiccant may vary depending on the desired properties of the pod. For more moisture removal the amount of the desiccant should be relatively large, for example at least 50%. If deodorizing is more important than moisture removal, the weight percentage of the solid carriers or scent may be larger. For example, a pod designed for deodorizing may contain 70% PEG or EVA, 20% fragrance and 10% SiO2.

The pod may also contain an amount of metallic spheres, for example stainless steel, to aid in the regeneration of the desiccating properties. The granules and particles may be coloured for aesthetic reasons.

The pod 10 may be of any shape necessary to be easily inserted and removed from a desired location, for example a shoe, glove, bag or locker. The pod may include an attachment device for attaching the pod to the desired location, such as means for hanging the pod within a locker. The pod may also simply be set in a desired location.

The pouch 12 should be shaped to have a sufficient surface area to allow moisture to enter the pod and fragrance to exit. Preferably the surface area to volume ratio of the pod will allow the desiccant granules to efficiently remove moisture from their surrounding environment. The pod 10 may be substantially flat. The shape of the pod 10 may be designed to allow physical contact of the anti-microbial fabric to a desired section of an article. The dimensions may be adjusted to fulfill the desired anti-microbial, desiccating and deodorizing effects.

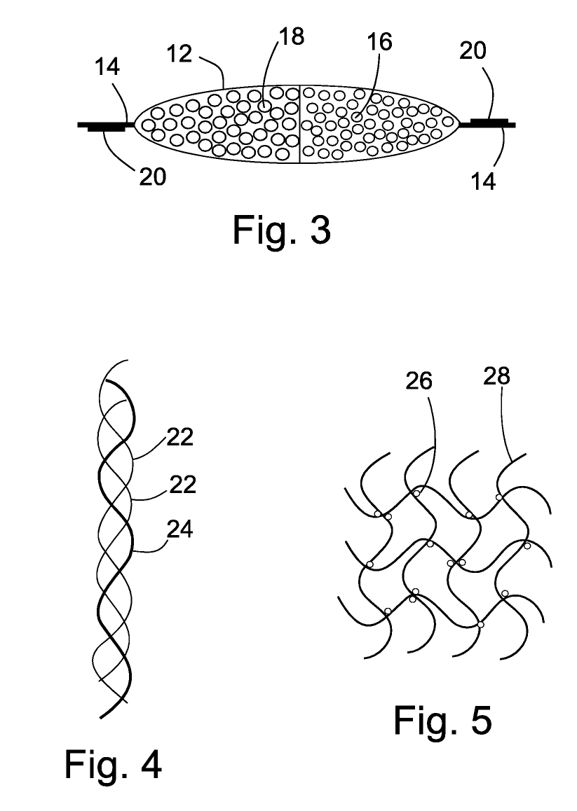

In an embodiment the pod 10 is a substantially rectangular shape. The pod 10 may be multi-compartmented as shown in FIG. 3, and the desiccant granules 16 and fragrance-releasing particles 18 may each be in separate compartments within the pouch 12.

The adsorbent pods 10 are reusable and may be used for a period of time before the moisture absorbing and deodorizing capacities are exhausted. The useful life of the pod is variable depending on the environment and conditions in which the pod is used. Even after the deodorizing capacity is depleted the anti-microbial properties continue to work and the moisture adsorbing properties may be recharged for further use. In an embodiment the pods may be used for up to 4 months before requiring a recharge. The moisture adsorbing properties of the pod may be recharged through thermal regeneration of the granules. For example, to recharge, the pod may be placed in a standard home clothes dryer and run at high heat for a period of 30 minutes or more. The pods may be recharged as necessary to maintain maximum moisture adsorption capabilities. The pod fabric should be chosen to be capable of withstanding multiple thermal regenerations.

In the claims, the word “comprising” is used in its inclusive sense and does not exclude other elements being present. The indefinite articles “a” and “an” before a claim feature do not exclude more than one of the feature being present. Each one of the individual features described here may be used in one or more embodiments and is not, by virtue only of being described here, to be construed as essential to all embodiments as defined by the claims.

The embodiments of the invention in which an exclusive property or privilege is claimed are defined as follows:

1. A pod for removing odor and moisture from a substantially confined area comprising:
a pouch formed from fabric, the fabric incorporating an antimicrobial agent; and
desiccant granules and fragrance-releasing particles confined within the pouch.

2. The pod of claim 1 in which the antimicrobial agent comprises silver.

3. The pod of claim 2 in which the silver is woven into the fabric.

4. The pod of claim 2 in which the silver is bonded to the fabric.

5. The pod of claim 1 in which the desiccant granules and the fragrance-releasing particles are separated into separate compartments within the pouch.

6. The pod of claim 1 in which the desiccant particles are thermally rechargeable.

7. The pod of claim 1 attached to one or more other pods, each of the one or more other pods comprising a pouch formed from fabric, the fabric incorporating an antimicrobial agent; and desiccant granules and fragrance-releasing particles confined within the pouch.

8. The pod of claim 1 in which the pouch contains a mixture of polyethylene glycol (PEG) 1-10 wt %, ethylene-vinyl acetate (EVA) 15-40 wt %, scent 10-25 wt %, silicon dioxide 30-50 wt %, metallic spheres 5-15 wt %, color 0-3 wt %.

9. The pod of claim 1 in which the pouch contains PEG in the range of 1-10% by weight.

10. The pod of claim 1 in which the pouch contains EVA in the range of 15-40% by weight.

11. The pod of claim 1 in which the pouch contains scent in the range of 10-25% by weight.

12. The pod of claim 1 in which the pouch contains silicon dioxide in the range of 30-50% by weight.

13. The pod of claim 1 in which the pouch contains metallic spheres in the range of 5-15% by weight.

14. The pod of claim 1 in which the pouch contains color in the range of 0-3% by weight.
